(19) United States
(12) Patent Application Publication
(10) Pub. No.: US 2010/0212667 A1
(43) Pub. Date: Aug. 26, 2010

(54) DRUG CAPSULES FOR DRY POWDER INHALERS

(75) Inventors: Simon James Smith, Hertford (GB); David Stuart Harris, Cambridge (GB)

Correspondence Address: CANTOR COLBURN, LLP, 20 Church Street, 22nd Floor, Hartford, CT, 06103, US

(73) Assignee: CAMBRIDGE CONSULTANTS LIMITED, Cambridge (GB)

(21) Appl. No.: 12/377,739

(22) PCT Filed: Aug. 16, 2007
(86) PCT No.: PCT/GB07/03128

§ 371 Date: May 12, 2010

(30) Foreign Application Priority Data
Aug. 16, 2006 (GB) 0616299.4

(51) Int. Cl. A61M 15/00; A61M 16/10; A61K 9/14; A61K 38/28; A61K 31/58; A61K 31/57; A61K 31/135; A61K 31/56; A61K 39/00

(52) U.S. Cl. 128/203.15; 141/9; 424/489; 514/3; 514/174; 514/179; 514/653; 514/178; 424/184.1

(57) ABSTRACT

A method of filling a drug capsule (2) for a dry powder inhaler comprising introducing a dose of powdered active substance (4) into the capsule and introducing a separate quantity of filler particles (6) into the capsule (2), said filler particles being of different composition and having a larger average particle size than the active substance.

This invention relates to capsules containing inhalable drugs for use with dry powder inhalers and to a method of filling them.

Dry powder inhalers are an increasingly common and effective way of delivering a variety of drugs by providing them in a sufficiently fine powder to allow them to be inhaled deep into the lungs and thereby pass into the bloodstream.

The micronised drug used with dry powder inhalers is often too fine to be useable on its own as an unacceptable proportion of the fine particles of the drug are deposited on the surfaces of the capsule or inhaler. It is therefore common to mix the fine drug particles with a suitably inert carrier substance of much larger particle size such as lactose. The drug particles then adhere to the carrier particles which allows them to be carried more easily through the inhaler. In addition, the larger carrier fraction makes the powder formulation more manageable in the manufacture of inhalers by improving flow ability. However this then gives rise to a need to de-aggregate the drug particles from the carrier in the inhaler, which it is difficult to achieve completely and so which can reduce the dose actually received by a user and increase the tendency for aggregated drug and carrier particles to hit the back of the user's throat which at the least causes an unpleasant sensation. It also increases production cost by requiring a mixing step which must ensure a homogeneous mix to allow accurate dosing.

It has been proposed to use a so-called ‘physical mixture’ of active substance and carrier particle in which there are no or substantially no adhesion forces between the two. Whilst this would reduce the need for de-aggregation in the inhaler, special measures are necessary during preparation to reduce adhesion (complete elimination being practically impossible) which adds to the cost of manufacture, and it is still necessary to ensure a homogenous mix in order that the active substance can be accurately dosed.

The present invention seeks to overcome the aforementioned problems and when viewed from a first aspect provides a method of filling a drug capsule for a dry powder inhaler comprising introducing a dose of powdered active substance into the capsule and introducing a separate quantity of filler particles into the capsule, said filler particles being of different composition and having a larger average particle size than the active substance. The invention also extends to a drug capsule filled in accordance with the aforementioned method. From one aspect this gives a capsule for use in a dry powder inhaler, containing an active substance and a filler substance wherein the majority of each powder is unmixed with the other.

Thus it will be seen by those skilled in the art that in accordance with the invention the powdered active substance is put into the capsule separately from the “filler” particles which allows the active substance to be measured accurately without having to ensure it is uniformly mixed with the filler particles. Indeed there is no deliberate mixing between the active substance and the carrier particles at all although some will inevitably occur e.g. as a result of agitation during transport. Such low level mixing will tend to form loosely bound agglomerates between particles of the two powders, but these can be easily broken down again in the inhaler.

When the two powdered substances are inhaled through a suitable dry powder inhaler, the larger, filler particles will act to scour the internal surfaces to dislodge any deposited active substance powder. This gives the benefits of a “pure” active substance, namely the ease of accurate dosing referred to above and also a reduction in the requirement for the inhaler to de-aggregate the powders. However, at the same time it prevents or substantially reduces the deposition of active substance on the internal surfaces of the inhaler.

The invention is of greatest benefit when used with dry powder inhalers which are highly selective in terms of the size of particles which are allowed to pass into the mouthpiece. Examples of such inhalers are disclosed in WO 2006/061637. These inhalers incorporate a reverse cyclone chamber (which term is defined therein) and a vortex finder which allows a highly selective passing of fine particles. The Applicant has observed that when used in conjunction with the present invention the two types of particles are entrained in the reverse cyclone flow with the smaller active substance particles circulating tightly in the forced vortex and therefore passing through the vortex finder with the larger particles being retained in the reverse cyclone chamber. However, the larger filler particles tend to scour the inner surface of the reverse cyclone chamber and therefore dislodge active substance particles which have become deposited on there. This results in a high proportion of the measured dose actually being received by the user and moreover the proportion is highly predictable. Thus a preferred application of the invention is a dry powder inhaler incorporating a reverse cyclone chamber but the invention may also be applied to other inhaler arrangements.

The capsule specified in accordance with the invention could be in the form of a replaceable pack such as a blister pack which is inserted into a suitable inhaler. Alternatively, the capsule could be comprised as part of an inhaler; for example it could be refillable or, more practically, the entire inhaler could be disposable. In either of the above possibilities the capsule preferably forms at least part of a circulating air flow chamber, most preferably a reverse cyclone chamber. In other words in accordance with at least some preferred embodiments of the invention the active substance and carrier powder are provided already in the circulation chamber which is used to give particle size selectivity.

Typically the active substance powder and the filler substance will be introduced into the capsule during production and the capsule then sealed. However, this is not essential. For example, the active substance and carrier could be stored in separate capsules and introduced separately into a separate chamber, e.g. an air circulation chamber by user immediately prior to use. Alternatively, one of the powders could be provided in the chamber with the other one being introduced by the user.

There are a wide variety of suitable active drug substances with which the invention may be used including, but not limited to inhalable insulin, budesonide, beclomethasone dipropionate, salbutamol, salmeterol, fluticasone propionate, inhalable vaccines. Similarly the filler particles could be any suitably inert substance including, but not limited to, lactose, mannitol, sucrose, glucose, trehalose or indeed any other sugars. Preferably the filler particles have a mass median aerodynamic diameter greater than 10 microns. Preferably the active substance has a mass median aerodynamic diameter less than 10 microns.

A preferred embodiment of the invention will now be described, by way of example only, with reference to the accompanying drawings in which:

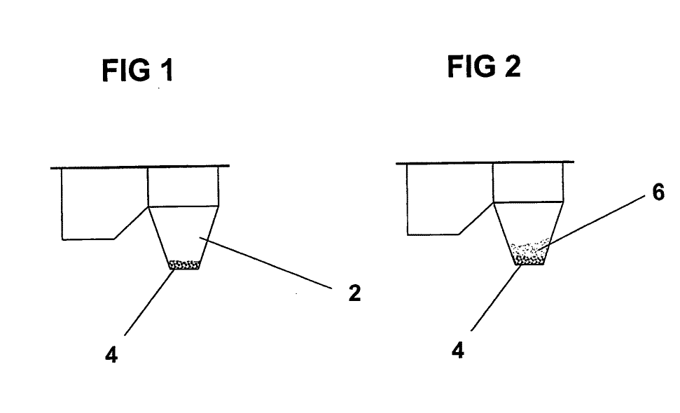

FIG. 1 is a schematic diagram of a first step of a capsule filling procedure in accordance with the invention;

FIG. 2 is a schematic diagram of a second step;

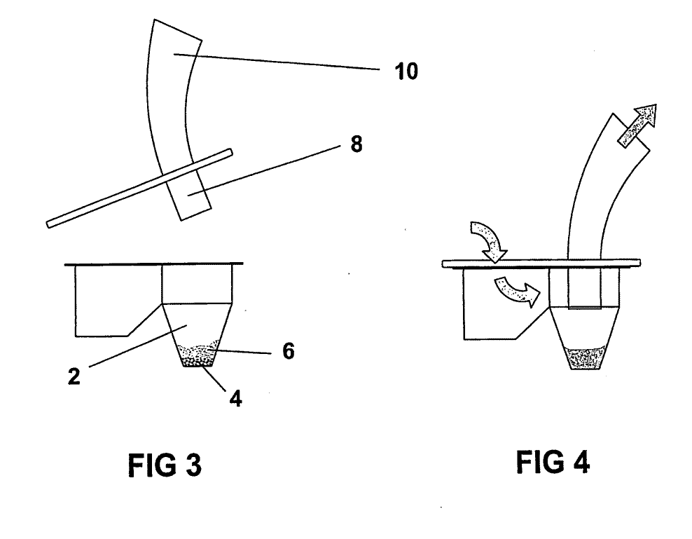

FIGS. 3 and 4 are schematic diagrams showing the capsule before and after installation into an inhaler;

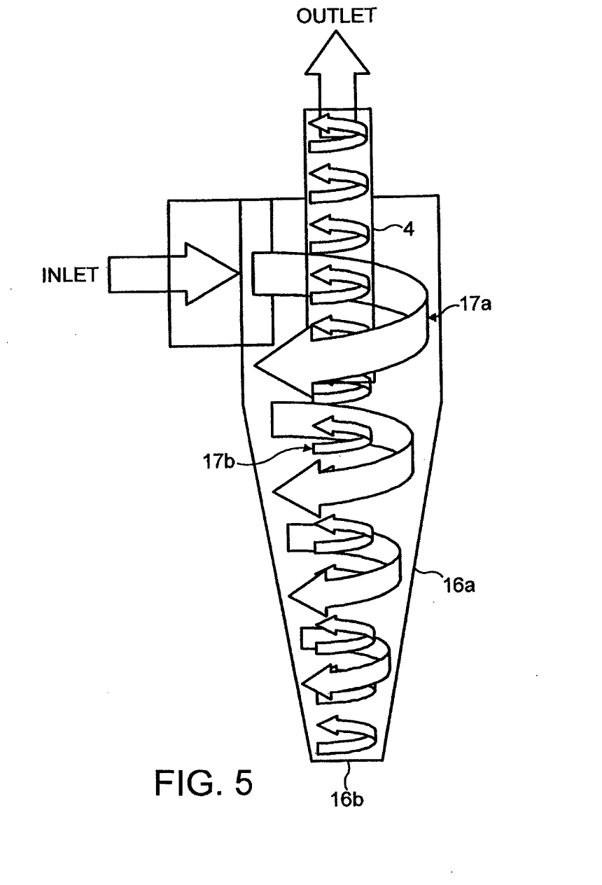

FIG. 5 is a schematic diagram showing the airflow in a reverse flow cyclone chamber; and

FIG. 6 is a graph showing the data measured during a test of an embodiment of the invention.

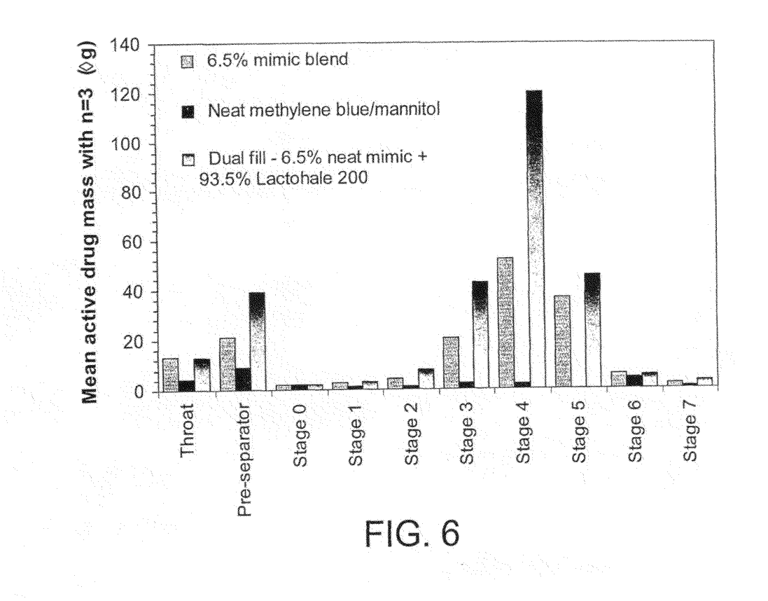

FIG. 1 shows schematically a blister pack capsule for the dry powder inhaler disclosed in the FIGS. 18 to 24 of WO 2006/061637. More particularly the blister comprises a moulded chamber portion 2 which is of cylindrical section in its upper portion and frusto-conical in its lower portion. To one side of the cyclone chamber 2 is an air inlet conduit (not shown) which allows air to be drawn tangentially into the cyclone chamber 2. During production of the blister pack an accurate dose of “pure”, i.e. unmixed, micronised drug is introduced into the chamber 2, as shown in FIG. 1. In the second stage, shown in FIG. 2, a filler substance such as lactose is introduced into the chamber 2 on top of the pure micronised drug. Thus it will be seen that quite contrary to the situation with known drug blister packs in which the drug and carrier substance (typically lactose) are mixed homogeneously, there is no deliberate mixing between the drug 4 and the coarse fraction 6. Even if some intermixing should take place through incidental agitation, e.g. during transport and handling, the resulting mixture will still be very clearly distinguishable from an artificial homogeneous mix. The loosely bound agglomerates formed will be easily broken up in the inhaler as described below.

The user uses the blister pack in exactly the same way as is described in WO 2006/061637. Thus the pack is placed inside the opened inhaler (represented in FIG. 3) and the inhaler closed (represented in FIG. 4 so that the piercer tube 8 which is formed at the bottom end of the mouthpiece 10 and which forms the vortex finder penetrates the foil membrane (not shown) of the blister. However, in accordance with the present invention the two powders, 4, 6 reside at the bottom of the chamber 2 and are entrained by the cyclone airflow which is set up.

The airflow upon inhalation by a user through the mouthpiece 10 is shown very generally in FIG. 4 and in more detail in FIG. 5. This airflow pattern is described in greater detail in WO 2006/061637 with reference to FIG. 6 thereof. However rather than the active substance having to be completely de-aggregated from a carrier, the airflow will naturally cause the filler particles 6 to tend to circulate around the walls of the chamber 2 in the boundary layer in the so-called free vortex 12, whilst the active substance is entrained upwardly in the forced vortex 14 to pass through the vortex finder and so into the mouthpiece. Any loosely bound agglomerates formed during incidental mixing between the active and filler will be broken up either by centrifugal forces in circulation or by the high shear force experienced between the inner and outer vortices.

The circulating filler particles tend to inhibit the deposition of the active substance on the walls of the chamber 2. This is enhanced by the fact that (as may be seen in FIG. 2) in the preferred embodiment the filler 6 is on top of the drug 4 and will therefore begin to circulate first. Furthermore, if any active substance is deposited on the walls of the chamber 2, the filler particles circulation will tend to dislodge it. The filler particles are large enough that, unlike the smaller active substance particles, they will not become trapped in the boundary layer of the circulating air. The filler particles will therefore experience an aerodynamic force from the airflow which will tend to re-entrain them even if they do become attached to the walls of the chamber

Some comparative tests were carried out to establish an example of the efficacy which can be achieved in accordance with the invention. In particular, the particle size distribution (by stage of penetration) of the aerosol emitted from a test inhaler was determined using an Anderson Cascade Impactor (USP Apparatus 3). The inhaler used for the test was as described in WO 2006/061637 with particular reference to FIGS. 18 to 24 thereof. The test apparatus was set to a flow rate of 32 liters per minute corresponding to a pressure drop of 4 kilopascals across the device. Three different formulations were tested, each being tested three times.

In the first formulation the blister capsule was filled with 12.8 milligrams of a mimic formulation. The mimic formulation was prepared according to known methods and thus 832 milligrams of active substance, methylene blue: mannitol with a mass median aerodynamic diameter (MMAD) of approximately 2 microns was homogeneously mixed with 11.968 mg of lactose (Lactohale 200) with an MMAD of approximately 70 microns.

In test 2 only the 832 milligrams of active substance was used without any lactose.

In test 3 the active substance was introduced first into the device and then the Lactohale 200 was filled on top of this without mixing. The quantities were the same as in test 1.

Each of the three tests was performed in triplicate and in accordance with the method described in USP 29 (601). As is well known in the art this comprises a portion simulating throat deposition, a pre-separator for collecting the large fraction and stages 0-7 corresponding to respective particle diameter ranges which represent varying degrees of penetration. The results of the tests are shown in the histogram in FIG. 6. The horizontal axis of this histogram shows the stages at which the active substance was deposited with the vertical axis representing the mean active drug mass in micrograms across the three repetitions of each test.

Considering firstly the leftmost column of each grouping of three, which represents the prior art blend of active substance and lactose, it can be seen that there is a good distribution of deposition between stages 3-6 (particularly stages 3-5) which is generally considered to be ideal. However, if this is compared to the neat drug distribution shown in the centre column of each group, it can be seen that there is very unfavourable distribution with most of the deposition taking place in the throat or pre-separator. Thus, despite the apparent advantage of just using the neat active substance, this is not practicable since it cannot be sufficiently effectively delivered.

However, if the third columns are considered, corresponding to filling in accordance with the invention, it may be seen that there is a very favourable distribution between stages 3 and 5 which, as mentioned above, is considered ideal. Thus it will be seen that at least this embodiment of the invention gives the dual advantages of a very favourable delivery distribution of the active particles as well as allowing for the accurate measurement of doses and obviating the need for a productions step of careful homogeneous mixing.

It will be appreciated that it is only examples of the invention that have been described and the invention may be employed with other combinations of active substance and filler. Moreover the invention need not be used with inhalers of the type described in WO 2006/061637, but could be used with other types of inhaler.

1. A method of filling a drug capsule for a dry powder inhaler comprising introducing a dose of powdered active substance into the capsule and introducing a separate quantity of filler particles into the capsule, said filler particles being of different composition and having a larger average particle size than the active substance.

2. A method as claimed in claim 2 comprising introducing the active substance powder and the filler substance into the capsule and then sealing the capsule.

3. A method as claimed in claim 1 wherein said powdered active substance comprises or more from the group comprising: inhalable insulin, budesonide, beclomethasone dipropionate, salbutamol, salmeterol, fluticasone propionate and inhalable vaccines.

4. A method as claimed in claim 1 wherein said filler particles comprise or more substances from the group comprising: lactose, mannitol, sucrose, glucose, trehalose and any other sugars.

5. A method as claimed in claim 1 wherein the active substance has a mass median aerodynamic diameter less than 10 microns.

6. A method as claimed in claim 1 wherein the filler particles have a mass median aerodynamic diameter greater than 10 microns.

7. A drug capsule filled in accordance with the method claimed in claim 1.

8. A capsule for use in a dry powder inhaler, containing an active substance and a filler substance wherein the majority of each powder is unmixed with the other.

9. A dry powder inhaler incorporating a capsule as claimed in claim 7.

10. A dry powder inhaler as claimed in claim 9 wherein the capsule forms at least part of a circulating air flow chamber.

11. A dry powder inhaler incorporating a reverse cyclone chamber and a capsule as claimed in claim 7.

12. A dry powder inhaler incorporating a capsule as claimed in claim 8.

13. A dry powder inhaler incorporating a reverse cyclone chamber and a capsule as claimed in claim 8.
